(19) United States
(12) Patent Application Publication
(10) Pub. No.: US 2025/0386987 A1
(43) Pub. Date: Dec. 25, 2025

(54) SURFACE CLEANING DEVICE WITH ODOR CONTROL

(71) Applicant: SHARKNINJA OPERATING LLC, Needham, MA (US)

(72) Inventors: Philippa J. COPELAND, Hayling Island (GB); Jemima L.C. MEIL, Lancaster (GB); Hugh J. CROGGON, Newbury (GB); Oliver G. HOLMES, London (GB)

(21) Appl. No.: 19/252,235

(22) Filed: Jun. 27, 2025

Related U.S. Application Data
(63) Continuation of application No. 17/980,700, filed on Nov. 4, 2022, now Pat. No. 12,364,374.
(60) Provisional application No. 63/276,151, filed on Nov. 5, 2021.

(51) Int. Cl. A47L 7/00; A47L 9/04; C11D 3/00; C11D 3/18; C11D 3/20

(52) U.S. Cl. CPC A47L 7/009; A47L 9/0477; C11D 3/0068; C11D 3/181; C11D 3/2086

(57) ABSTRACT

A surface cleaning device may include a nozzle, a dust cup. a suction motor configured to draw air into the nozzle and through the dust cup, and a first deodorizer coupled to the nozzle, the first deodorizer includes a deodorizing composition having a long chain fatty acid.

### CROSS-REFERENCE TO RELATED APPLICATIONS

The present application is a continuation of U.S. patent application Ser. No. 17/980,700 filed Nov. 4, 2022, which claims the benefit of U.S. Provisional Application Ser. No. 63/276,151 filed on Nov. 5, 2021, entitled Surface Cleaning Device with Odor Control, both of which are incorporated herein by reference.

### TECHNICAL FIELD

The present disclosure is generally directed to surface cleaning devices and, more particularly to, a surface cleaning device including a deodorizer.

### BACKGROUND INFORMATION

The following is not an admission that anything discussed below is part of the prior art or part of the common general knowledge of a person skilled in the art.

Powered surface cleaning devices, such as vacuum cleaners, have multiple components that each receive electrical power from one or more power sources (e.g., one or more batteries or electrical mains). For example, a vacuum cleaner may include a suction motor to generate a vacuum within a nozzle. The generated vacuum collects debris from a surface to be cleaned and deposits the debris, for example, in a dust cup. The vacuum may also include a motor to rotate a brush roll within the nozzle. The rotation of the brush roll agitates debris that has adhered to the surface to be cleaned such that the generated vacuum is capable of removing the debris from the surface. In addition to electrical components for cleaning, the vacuum cleaner may include one or more light sources to illuminate an area to be cleaned.

### BRIEF DESCRIPTION OF THE DRAWINGS

These and other features advantages will be better understood by reading the following detailed description, taken together with the drawings wherein:

FIG. 1 shows an example surface cleaning device consistent with aspects of the present disclosure.

FIG. 2 shows an example nozzle suitable for use within the surface cleaning device of FIG. 1.

FIG. 3 shows a cross-sectional view of a nozzle suitable for use within the surface cleaning device of FIG. 1 consistent with aspects of the present disclosure.

FIG. 4A shows an example deodorizer consistent with aspects of the present disclosure. FIG. 4B shows the deodorizer being installed within a nozzle and may be an example of the deodorizer and nozzle of FIG. 2.

FIG. 5A shows another example nozzle of a surface cleaning device implemented with a deodorizer consistent with aspects of the present disclosure.

FIG. 5B shows a side view of a nozzle coupled to a deodorizer consistent with aspects of the present disclosure.

FIG. 5C shows an example disposable deodorizer consistent with aspects of the present disclosure.

FIG. 6 shows a plurality of example deodorizers consistent with aspects of the present disclosure.

FIG. 7 is a block diagram that maps origins for malodors within a home/office and associated molecule(s) that can cause the malodor.

FIG. 8 shows an example table that shows origins for malodors within a home/office and associated molecule(s) that can cause the malodor.

FIG. 9 shows the chemical formula of stearic acid.

FIG. 10 shows an example of a compound suitable for use as a deodorizing compound consistent with aspects of the present disclosure.

FIG. 11 shows an example deodorizer consistent with aspects of the present disclosure.

FIG. 12 shows another example deodorizer consistent with aspects of the present disclosure.

FIG. 13 is a table enumerating various example compositions for use as or in a deodorizer consistent with aspects of the present disclosure.

FIG. 14 is a table demonstrating relative durability between a plurality of different configurations for a deodorizer consistent with aspects of the present disclosure.

The drawings included herewith are for illustrating various examples of articles, methods, and apparatuses of the teaching of the present specification and are not intended to limit the scope of what is taught in any way. Like reference numerals refer to like parts throughout the various views unless otherwise specified.

### DETAILED DESCRIPTION

The present disclosure is generally directed to a surface cleaning device configured to include a deodorizer for odor control/conditioning during operation of the surface cleaning device. The deodorizer may include a compound formed of a long chain fatty acid such as stearic acid. The deodorizer can be coupled to the surface cleaning device at a location where the deodorizer is agitated during operation of the surface cleaning device. For example, the deodorizer can be positioned to (directly) engage/contact a surface to be cleaned such that friction therebetween causes particles of the deodorizer (e.g., particles formed of long chain fatty acid molecules) to be released from the deodorizer. Alternatively, or in addition, the deodorizer can be positioned to (directly) engage/contact a cleaning element (e.g., a rotating agitator such as a brush roll or foam roller) such that contact between the cleaning element and the deodorizer causes particles of the deodorizer to be released from the deodorizer.

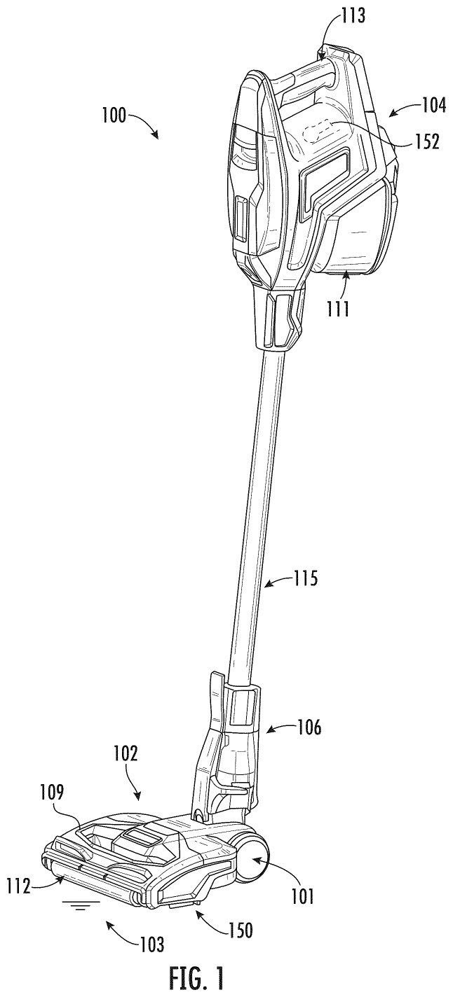

FIG. 1 shows an example surface cleaning device 100. As shown, the surface cleaning device 100 is illustrated as stick-type vacuum. However, aspects and features of the present disclosure may also be implemented in other vacuum types including, for example, hand-held vacuums, robotic vacuums, upright vacuums, cannister vacuums, and/or any other type of vacuum cleaner.

As shown, the surface cleaning device 100 includes a nozzle 102, a motor housing 104 having a suction motor 152, a dust cup 111, and an upright section 106. The nozzle 102 includes a nozzle housing 109, one or more wheels 101, at least one cleaning element 112 (e.g., a brush roll or foam roller), and a dirty air inlet 150. The one or more wheels 101 are configured to enable a user to roll the nozzle 102 across a surface to be cleaned 103 (e.g., according to a push/pull motion). In some instances, the nozzle 102 may include a plurality of cleaning elements 112 configured to rotate, wherein at least one cleaning element 112 is different from at least one other cleaning element 112. For example, a first cleaning element 112 may be a brush roll and a second cleaning element 112 may be a foam roller.

The dirty air inlet 150 is fluidly coupled to a dirty air passageway. The dirty air passageway can be defined, at least in part, by the nozzle housing 109, the upright section 106, a conduit/wand 115, and the dust cup 111. The dust cup 111 can be removably coupled to the motor housing 104. The suction motor 152 is configured to cause air having debris entrained therein to flow along the dirty air passageway. In other words, the suction motor 152 is configured to draw air into the nozzle 102 and through the dust cup 111. At least a portion of the entrained debris may fall out of entrainment when the air is passing through the dust cup 111 and be collected within the dust cup 111 for later disposal.

The at least one cleaning element 112 is configured to (directly) engage the surface to be cleaned 103 during cleaning operations. The at least one cleaning element 112 can be configured to rotate about a longitudinal axis of the rotatable cleaning element 112. The at least one cleaning element 112 may be configured to be driven via a brush roll motor (not shown) and/or can be configured to be driven via manual movement based on a user supplying a push/pull force to the surface cleaning device 100 via a handle 113.

The at least one cleaning element 112 can be configured to (directly) engage a deodorizer consistent with the present disclosure, as discussed in further detail below.

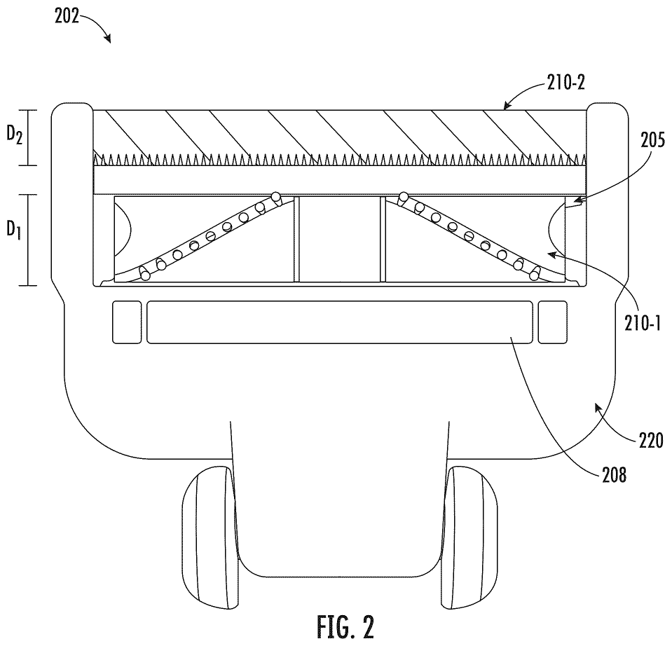

FIG. 2 shows a schematic example of a nozzle 202. The nozzle 202 is an example of the nozzle 102 of FIG. 1. As shown, the nozzle 202 includes a bottom surface 220 that faces a surface to be cleaned (e.g., the surface to be cleaned 103 of FIG. 1), first and second cleaning elements 210-1 and 210-2, and a dirty air inlet 205. The dirty air inlet 205 is at least partially defined in the bottom surface 220. In operation, at least a portion of one or more of the first and second cleaning elements 210-1 and 210-2 may extend from the dirty air inlet 205 in a direction of the surface to be cleaned.

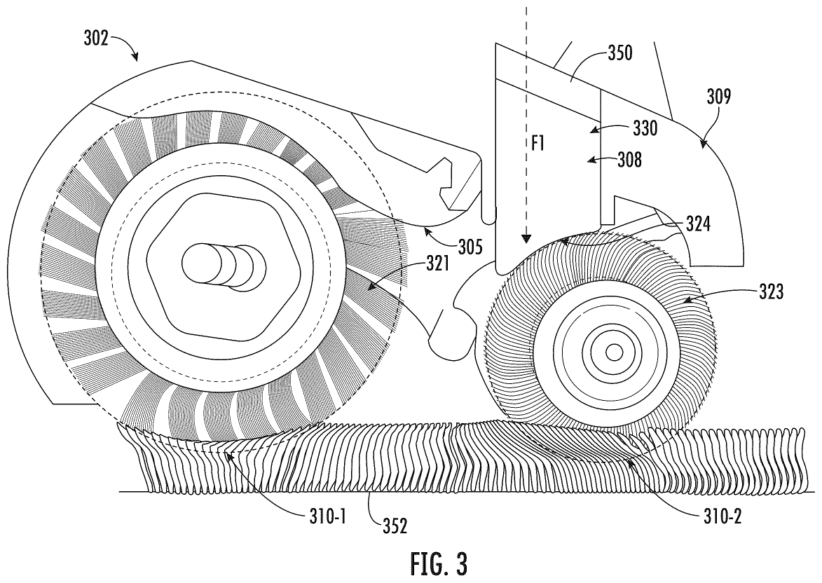

The first cleaning element 210-1 can extend substantially parallel with the second cleaning element 210-2. The first cleaning element 210-1 may be a first type of cleaning element and the second cleaning element 210-2 may be a second type of cleaning element, with the first and second types of cleaning elements being different. For example, the first cleaning element 210-1 can be a brush roll having bristles to engage the surface to be cleaned, such as the bristles 321 shown in FIG. 3, and the second cleaning element 210-2 can be a foam roller to engage the surface to be cleaned, such as the foam roller 323 shown in FIG. 3.

The first cleaning element 210-1 may include a first material such as thermoplastic. In one example, the first cleaning element 210-1 includes relatively thin bristles formed of a material such as nylon. The diameter of the bristles may be in a range of 0.04±0.02 mm. The second cleaning element 210-2 may include a second material such as thermoplastic with nylon bristles, for example.

The first cleaning element 210-1 may have an overall diameter DI that is greater than an overall diameter D2 of the second cleaning element 210-2. The first cleaning element 210-1 may have an overall diameter in a range of 15 millimeters (mm) to 30 mm. The second cleaning clement 210-2 may have an overall diameter in a range of 30 mm to 60 mm.

While the nozzle 202 is shown as including a first and second cleaning element 210-1 and 210-2, other configurations are possible. For example, the nozzle 202 may include only one of the first or second cleaning elements 210-1 or 210-2. By way of further example, the nozzle 202 may include one or more additional cleaning elements in addition to the cleaning elements 210-1 and 210-2.

The nozzle 202 may further include a deodorizer 208 coupled to the bottom surface 220. The deodorizer 208 can extend in parallel with the first cleaning element 210-1 and/or the second cleaning element 210-2.

The deodorizer 208 extends from the bottom surface 220 in a direction of the surface to be cleaned by an extension distance. The extension distance may be, for example, in a range between 0 mm and 20 mm. The extension distance may be greater than or equal to the overall distance that the first cleaning clement 210-1 and/or the second cleaning element 210-2 extend from the bottom surface 220 of the nozzle 202. Such a configuration may encourage the deodorizer 208 to (directly) engage with the surface to be cleaned during cleaning operations. This configuration may also be referred to herein as a direct application configuration.

FIG. 3 shows a cross-sectional view of a nozzle 302, which is an example of the nozzle 102. As shown, the nozzle 302 includes a nozzle housing 309, a first cleaning element 310-1 implemented as a brush roll having bristles 321 extending therefrom, and a second cleaning element 310-2 implemented as a foam roller which is configured to (directly) engage a deodorizer 308. This configuration may also be referred to herein as an indirect applicator configuration. Alternatively, or in addition, the deodorizer 308 can be configured to extend to a distance that causes the deodorizer 308 to also contact/engage (directly) a surface to be cleaned. This configuration may be referred to herein as a dual applicator configuration.

As shown, the deodorizer 308 is disposed in a cavity/receptacle 330 defined by the nozzle housing 309. A biasing mechanism 350 (e.g., a spring) may be disposed within the cavity/receptacle 330 at a location between the nozzle housing 309 and the deodorizer 308. The biasing mechanism 350 may be configured to supply a force Fl that urges the deodorizer 308 in a direction of the second cleaning clement 310-2. For example, the force F1 may extend transverse to (e.g., substantially perpendicular to) a surface to be cleaned 352. Such a configuration may allow the deodorizer 308 to remain in (direct) engagement with the second cleaning element 310-2. In some instances, a weight of the deodorizer 308 may be such that the deodorizer 308 remains in (direct) engagement with the second cleaning element 310-2. Such a configuration may omit the biasing mechanism 350.

As shown, the deodorizer 308 defines an engagement surface 324 for (directly) engaging an outer surface of the second cleaning element 310-2. The engagement surface 324 can include a shape/profile that corresponds with the shape/profile of the second cleaning element 310-2. The engagement surface 324 can include this shape/profile when manufactured, or the engagement surface 324 can be introduced via mechanical friction caused between the deodorizer 308 and the second cleaning element 310-2.

The deodorizer 308 is configured to release particles of a deodorizing composition based on the engagement surface 324 (directly) engaging the outer surface of the second cleaning element 310-2. The released particles are configured to couple to the outer surface of the second cleaning clement 310-2 and/or be released into the surrounding area. In either case, released particles may then be introduced/transferred to the surface to be cleaned 352. Alternatively, or in addition, released particles may be drawn into the dirty air passageway 305. This can reduce malodors within the dust cup, and also allow for released particles to be exhausted via an outlet of the surface cleaning device.

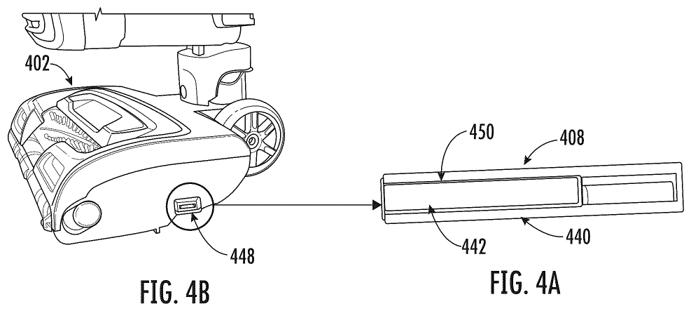

FIG. 4A shows an example deodorizer 408 consistent with aspects of the present disclosure. The deodorizer 408 may be one example of the deodorizer 208 of FIG. 2 and/or the deodorizer 308 of FIG. 3. FIG. 4B shows the deodorizer 408 being installed within a nozzle 402 and may be an example of the deodorizer 208 and nozzle 202 of FIG. 2.

The deodorizer 408 includes a cartridge body 440 configured to removably couple with the nozzle 402 and a deodorizing composition (or deodorant) 442 coupled to the cartridge body 440. For example, the cartridge body 440 may define a cavity/receptacle 450 configured to receive at least a portion of the deodorizing composition 442. The deodorizing composition 442 may be configured to extend from an open end of the cavity/receptacle 450. The cartridge body 440 may be formed from a material such as a thermoplastic, e.g., acrylonitrile butadiene styrene (ABS).

As shown, the cartridge body 440 may have a length that is equal to or greater than half of a length of a cleaning element rotatably coupled to the nozzle 402 (e.g., the first and/or second cleaning elements 210-1, 210-2, 310-1, and/or 310-2 of FIGS. 2 and 3). Such a configuration may allow the deodorizer 408 to engage (directly) at least half of the cleaning element. For example, the cartridge body 440 may have an elongated profile (e.g., having a length that is greater than a width).

The deodorizing composition 442 includes one or more odor reducing materials, such as but not limited to one or more long chain fatty acids. In examples, the odor reducing material(s) are or include a C12-C20 fatty acid, such as stearic acid (a C18 fatty acid) as discussed in further detail below.

As shown in FIG. 4B, the nozzle 402 includes a cartridge receptacle 448. The cartridge receptacle 448 is configured such that the deodorizer 408 can be slidably inserted therein. The cartridge receptacle 448 can be configured to align the deodorizer 408 with a surface to be cleaned such as shown and described above regarding FIG. 2. The cartridge receptacle 448 may also be disposed at other locations along the nozzle such as at a position that aligns the inserted deodorizer 408 with an associated cleaning element, e.g., the second cleaning element 310-2 as shown in FIG. 3. In one example, a nozzle can include a plurality of cartridge receptacles 448, with each cartridge receptacle 448 configured to align an inserted deodorizer 408 with an associated cleaning element and/or the surface to be cleaned. Alignment of the deodorizer refers to the deodorizer 408 being disposed at a location that causes the deodorizer 408 (e.g., the deodorizing composition 442) to be (directly) engaged/contacted by the associated cleaning element and/or surface to be cleaned.

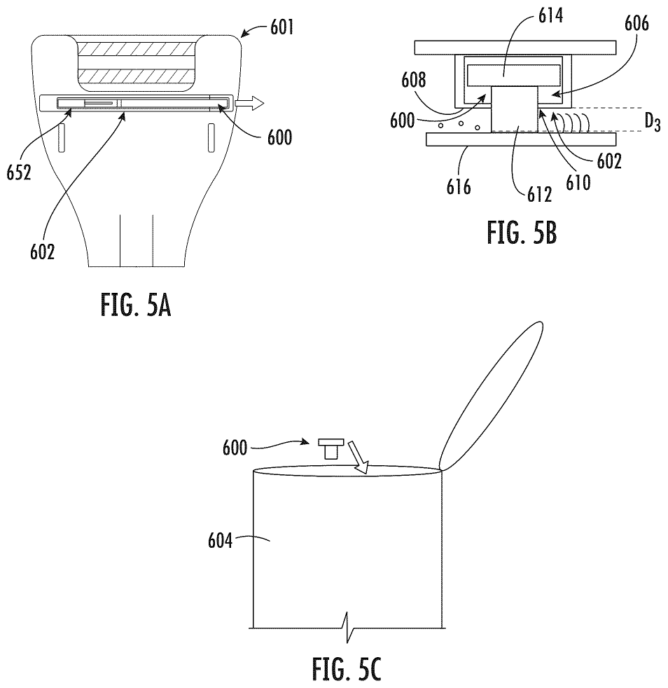

FIGS. 5A and 5B show an example of a deodorizer 600, wherein a portion of the deodorizer is configured to be selectively received within a receptacle 602 of a nozzle 601. The deodorizer 600 is an example of the deodorizer 408 of FIG. 4 and the receptacle 602 is an example of the cartridge receptacle 448 of FIG. 4. FIG. 5C shows the deodorizer 600 removed from the receptacle 602 (e.g., for disposal in a trash receptacle 604).

As shown in FIGS. 5A and 5B, the deodorizer 600 can be selectively received within the receptacle 602. As shown, the receptacle 602 defines a receiving region 606. The receiving region 606 includes retaining flanges 608, wherein a passthrough 610 extends between the retaining flanges 608. When the deodorizer 600 is received within the receptacle 602 at least a portion of the deodorizer 600 (e.g., a deodorizing composition, or deodorant, 612 of the deodorizer 600) extends through the passthrough 610 and the retaining flanges 608 may (directly) engage at least a portion of the deodorizer 600 (e.g., a cartridge body 614 of the deodorizer 600) to retain at least a portion of the deodorizer 600 within the receptacle 602. As shown, the deodorizing composition 612 extends through the passthrough 610 and into (direct) engagement with a surface to be cleaned 616. For example, the deodorizing composition 612 may extend from the passthrough by an extension distance D3. The extension distance D3 may be in a range of, for example, 0 mm and 20 mm.

In some instances, a plurality of deodorizers 600 may be coupled to the nozzle 601. For example, the nozzle 601 may include a plurality of receptacles 602, each configured to receive a respective deodorizer 600. At least one of the plurality of deodorizers 600 may be configured to (directly) engage the surface to be cleaned (or a cleaning element). For example, a first receptacle 602 may be configured to position a first deodorizer 600 to (directly) engage a surface to be cleaned (e.g., similar to as shown in FIG. 2) and a second receptacle 602 may be configured to position a second deodorizer 600 to (directly) engage a cleaning element (e.g., similar to as shown in FIG. 3).

The deodorizer 600 can be removed from the receptacle 602. For example, the receptacle 602 may include a latch 652, wherein, in response to actuation of the latch 652, the deodorizer 600 can be removed from the receptacle 602. The latch 652 may be configured to transition between a retaining position and a release position, wherein, when in the retaining position, a portion of the latch 652 is (directly) engaging the deodorizer 600. In some instances, the latch 652 can be biased towards the retaining position.

As shown in FIG. 5C, in response to actuation of the latch 652, the deodorizer 600 can be removed from the receptacle and disposed of by a user. A replacement deodorizer may then be inserted into the receptacle 602. The deodorizer 600 is configured to be disposed of after a predetermined period of use. The predetermined period of use may be, for example, in a range of 10 to 30 operating hours. By way of further example, the predetermined period of use may be at least 15 operating hours.

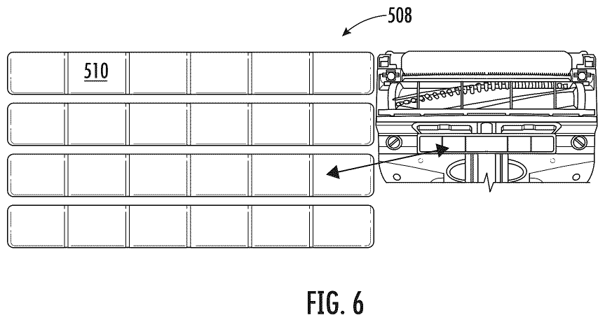

FIG. 6 shows a plurality of example deodorizers 508, which are another example of the deodorizer 208 of FIG. 2. In this example, the deodorizer 508 has an engaging surface 510 and a coupling surface opposite the engaging surface 510. The engaging surface 510 is configured to (directly) engage a surface to be cleaned and the coupling surface is configured to removably couple the deodorizer 508 to a nozzle of a surface cleaning device (e.g., using an adhesive, hook and loop fastening, and/or any other form of removable coupling).

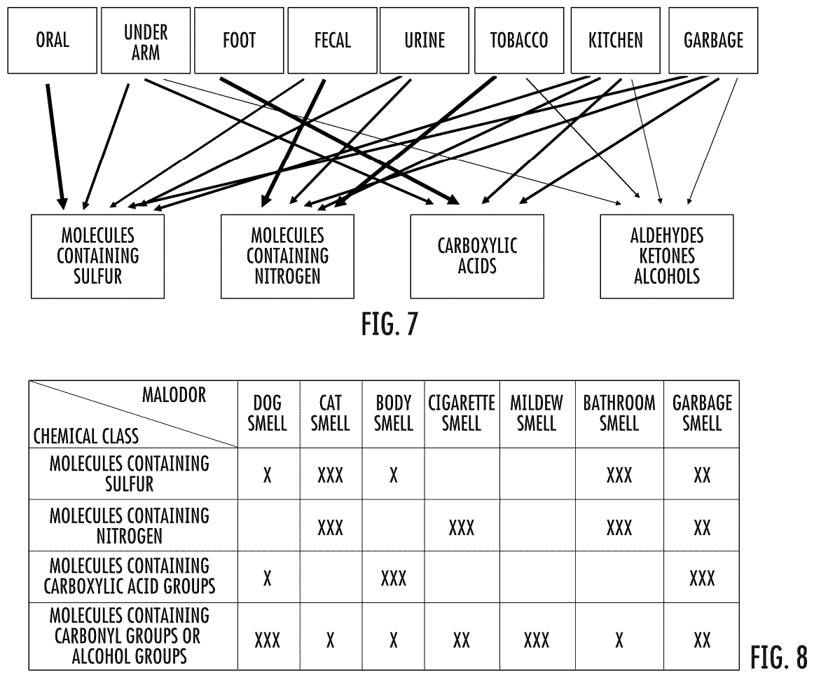

One aim of the present disclosure is odor management of malodors commonly present in homes. Some such odors include those caused by molecules containing sulfur, molecules containing nitrogen, molecules containing carboxylic acids, and/or molecules containing carbonyl groups (e.g., aldehydes, ketones) or alcohol groups. FIG. 7 graphically maps example origins of malodor and molecule(s) giving rise to those malodors. FIG. 8 is a table that enumerates malodor origins and chemical classifications giving rise to those malodors.

One aspect of the present disclosure has identified compounds formed from long chain fatty acids are particularly well suited for odor control, and in particular, those odors attributable to molecules containing sulfur, nitrogen and/or carbonyl groups. Long chain fatty acids are also negatively charged and can provide an anti-static agent. As used herein, “long chain fatty acid” means a saturated or unsaturated fatty acid having from 12 to 22 carbon atoms, and preferably from 16 to 22 carbon atoms. The long chain fatty acids described herein are saturated and contain from 12 to 22 (or 16 to 22) carbon atoms.

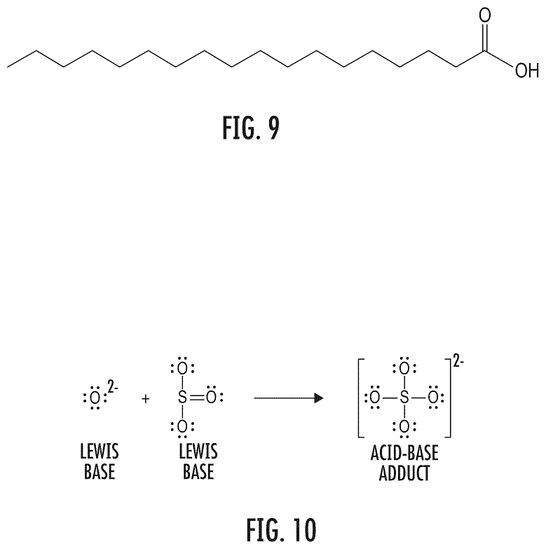

Experimental results have demonstrated that stearic acid is particularly well suited for minimizing or otherwise reducing malodors from surfaces such as carpets. Indeed, the inventors have found that application of stearic acid to a surface such as a carpet tends to soften carpet fibers as well as mitigate/prevent static buildup. FIG. 9 shows an example chemical composition of stearic acid and how the same has a carboxylic acid group head and a long hydrocarbon chain tail.

With the foregoing in mind, one aspect of the present disclosure relates to deodorizers for use with cleaning devices, and surface cleaning devices including the same. The deodorizers described herein may be formed from or include (e.g., the deodorizing composition 442, 612 includes) a deodorizing composition. The deodorizing composition may be formed from or include a deodorizing agent, either alone or in combination with a carrier material and one or more optional additives.

Non-limiting examples of suitable deodorizing agents include long chain fatty acids (e.g., stearic acid), Lewis acid-base adducts (sec, FIG. 10), a metallic salt such as aluminium chlorohydrate, sodium bicarbonate, zinc ricinoleate (Zn²⁺), combinations thereof, and the like.

In one example, the deodorizing agent consists, consists essentially of, or includes a long chain fatty acid, such as but not limited to stearic acid. In other examples, the deodorizing agent consists, consists essentially of, or includes a metallic salt, such as but not limited to zinc ricinoleate. And, in still further examples, the deodorizing agent consists, consists essentially of, or includes a combination of a long chain fatty acid and a Lewis acid-base adduct.

In examples, the deodorizing composition includes a first deodorizing agent and optionally a second deodorizing agent. In such instances, the first deodorizing agent may be or include a long chain fatty acid such as stearic acid, and the optional second deodorizing agent may be configured to target removal of odors resulting from carboxylic acid containing compounds, e.g., metallic salt such as zinc ricinoleate. In some instances, the second deodorizing agent may generally be referred to as an odor control compound. The odor control compound may be, for example, zinc ricinoleate.

Without limitation, in examples the deodorizing composition includes a combination of stearic acid and zinc ricinoleate. The stearic acid provides a first section of a deodorizer and the zinc ricinoleate provides a second section of a deodorizer, with the first and second sections being coupled to each other to form the deodorizing composition. The relative amounts of first and second deodorizing agents in the deodorizing composition may be selected to achieve desired deodorization performance.

For example, the first and second deodorizing agents may each be present in the deodorizing composition in an amount ranging from greater than 0 to less than 100% by weight (weight %) of the deodorizing composition. In examples, the first deodorizing agent (e.g., a fatty acid such as stearic acid) is present in the deodorizing composition in an amount ranging from about 50 to about 99 weight % (e.g., from about 80 to about 99 weight % or from about 90 to about 99 weight %), and the second deodorizing agent (e.g., a metallic salt such as zinc ricinoleate) is present in the deodorizing composition in an amount ranging from greater than 0 to about 20 weight % (e.g., from about 0.5 to about 5 weight % or from about 0.5 to about 2.5 weight %). In examples, the first deodorizing agent (e.g., a fatty acid such as stearic acid) is present in the deodorizing composition in an amount ranging from about 50 to 100 weight % and the second deodorizing agent (e.g., a metallic salt such as zinc ricinoleate) is present in the deodorizing composition in an amount ranging from greater than 0 to about 20 weight %. In examples, the first and second deodorizing agents are present in the deodorizing composition in the above noted amounts and the total amount of the first and second deodorizing agents (either alone or in combination with, for example, a carrier material, if used) equal 100% by weight of the deodorizing composition.

The carrier material is generally configured to adjust the physical properties of the deodorizing composition, and may be selected to achieve a deodorizing composition that can be applied to carpet or fabric without breaking. Non-limiting examples of suitable carrier materials that may be used include soy wax, cetearyl alcohol, paraffin wax, Microcrystalline wax, combinations thereof, and/or the like. In some examples, the carrier material is soy wax, paraffin wax, or a combination thereof.

When used, the carrier material may be present in the deodorizing composition in an amount ranging from greater than 0 to about 99 weight %, such as from greater than 0 to about 90 weight %, greater than 0 to about 80 weight %, or from about 10 to about 80 weight %. In examples, the deodorizing composition includes a first and/or second deodorizing agent in the above noted weight % ranges, with the carrier material making up the balance of the deodorizing composition.

In specific examples, the deodorizing composition includes a combination of a long chain fatty acid such as stearic acid as a deodorizing agent and soy wax or paraffin wax as a carrier material. In such instances, the relative amounts of long chain fatty acid and carrier material may be adjusted to achieve desired physical properties. For example, increasing the amount of fatty acid (e.g., stearic acid) in the deodorizing composition (while decreasing the amount of carrier material) may increase the brittleness of the deodorizing composition, while increasing or decreasing the durability of the deodorizer. In contrast, increasing the amount of carrier material relative to the amount of fatty acid may result in a deodorizing composition that is more easily spread on carpet and/or fabric, but may reduce the durability or useful lifetime of the deodorizer. In examples, the deodorizing composition includes a fatty acid as a deodorizing agent, but does not include a carrier material. In other examples, the deodorizing composition includes a fatty acid as a deodorizing agent and includes a carrier material, where a ratio of fatty acid to carrier material is in a range of, for example, 1:9 to 4:1 or 1:4 to 2:2.

As noted above, the deodorizing compositions described herein may include one or more optional additives, which may be selected to provide or enhance certain performance characteristics of the deodorizing composition. Surfactants, fragrance fixatives, and low odor fragrances (odor level 1.0 or less) are non-limiting examples of optional additives that may be included in the deodorizing composition.

Non-limiting examples of suitable surfactants that may be used include surfactants that increase the substantivity of the deodorizing composition, i.e., the degree to which the deodorizing composition sticks to fabric/carpet. Non-limiting examples of suitable surfactants that can increase or modify the substantivity of the deodorizing composition include cationic surfactants such as dipalmitoylethyl hydroxyethylmonium methosulfate, and/or other quaternary ammonium compounds such as benzethonium chloride, combinations thereof, and the like.

When used, such surfactants may be included in the deodorizing composition.

Without limitation, in examples the deodorizing compositions described herein include a cationic surfactant (e.g., dipalmitoylethyl hydroxyethylmonium methosulfate) in an amount ranging from about 0.01 to about 90 weight %.

Non-limiting examples of fragrance fixatives that may be included in the deodorizing composition include triethyl citrate or diphenylmethane. When used, such fragrance fixatives may be included in the deodorizing composition in an amount ranging from equal to 0.01% to about 20%.

In some examples, deodorizers consistent with the present disclosure include a reinforcement member that can be embedded within or impregnated with the deodorizing composition. The reinforcement member may be used to provide additional structural integrity or other desired physical characteristics.

Non-limiting examples of suitable reinforcement members that can be used include woven and non-woven materials, such as woven and non-woven sheets (e.g., dryer sheets). In non-limiting examples, the deodorizers described herein include a reinforcement member that is formed from or includes a woven or non-woven sheet (e.g., a dryer sheet) that is embedded within or impregnated with other elements of the deodorizing composition described herein. For example, the deodorizer can include a reinforcement member in the form of non-woven (e.g., dryer sheet) that is impregnated with a first and/or second deodorizing agent (e.g., stearic acid, zinc ricinoleate, and combinations thereof), either alone or in combination with one or more optional additives discussed above.

A Lewis acid or electrophilic compound can be added to target molecules containing carboxylic acids. A Lewis acid can be used to counteract malodors by creating an acid-base adduct, such as shown in FIG. 10. FIG. 10 shows an example of an electrophile (also known as a Lewis acid) and a nucleophile (also known as a Lewis base) covalently bonding to form a compound.

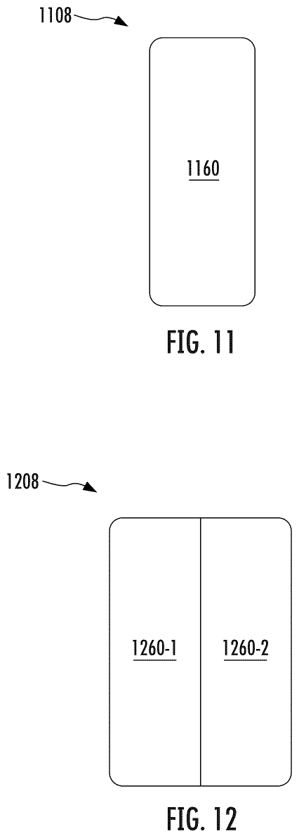

FIG. 11 shows an example deodorizer 1108 consistent with aspects of the present disclosure. The deodorizer 1108 can be an example of the deodorizer 208 of FIG. 2. For example, the deodorizer 1108 can be disposed on a bottom surface of a nozzle and can be configured to engage (directly) a surface to be cleaned to release particles for odor management. Alternatively, or in addition, the deodorizer 1108 can be an example of the deodorizer 308 of FIG. 3. For example, the deodorizer 1108 can be configured to engage (directly) an associated cleaning element, such as the second cleaning element 310-2 of FIG. 3.

The deodorizer 1108 may include a first section 1160 formed from a deodorizing composition as noted above. In examples where the deodorizer 1108 consists of the first section 1160, it may be referred to as a single homogenous strip/bar.

In examples, the first section 1160 is formed from (e.g., consists or consists essentially of) or includes a long chain fatty acid such as stearic acid. In specific examples, first section 1160 includes stearic acid, the chemical composition of which is shown in FIG. 9.

In other examples, the first section 1160 includes a fatty acid such as stearic acid, in combination with an acid-base adduct such as zinc ricinoleate. The first section 1160 can define an engagement surface, such as engagement surface 324 of FIG. 3.

The deodorizing composition may be formed in any suitable manner. For example, when the deodorizing composition includes first and second deodorizing agents, formation of the deodorizing composition may include melting the first and second deodorizing agents and then combining the melts with one another.

Alternatively, the first and second deodorizing agents may be mixed within one another (e.g., as solid particulates) and then melted together to form a melt containing both the first and second deodorizing agents. The carrier and optional additives noted above can be combined with the deodorizing agent(s) in any suitable manner, e.g., by melting them separately or with the deodorizing agent(s). When the deodorizing agent(s) and other components are combined to form a melt, the melt may be molded into a form that can be used as or in the deodorizer 1108. Alternatively, or additionally, when a reinforcement member is used, the reinforcement member may be embedded in or impregnated with the melt in any suitable manner. In such instances, the reinforcement member may be selected to provide additional structural integrity/strength to the deodorizer 1108.

The deodorizer 1108 may have an overall volume in a range of 20 milliliters (ml) to 80 ml. The deodorizer 1108 may have an overall height in a range of 5 mm to 25 mm, an overall length in a range of 100 mm to 280 mm and an overall thickness in a range of 5 mm to 25 mm.

FIG. 12 shows another example deodorizer 1208 consistent with the present disclosure. The deodorizer 1208 can be an example of the deodorizer 208 of FIG. 2. For example, the deodorizer 1208 can be disposed on a bottom surface of a nozzle to engage (directly) a surface to be cleaned to release particles for odor management. Alternatively, or in addition, the deodorizer 1208 can be an example of the deodorizer 308 of FIG. 3. For example, the deodorizer 1208 can be configured to engage (directly) an associated cleaning element, such as the second cleaning element 310-2 of FIG. 3.

The deodorizer 1208 includes a first portion/section 1260-1 formed from a first material and a second portion 1260-2 formed from a second material, wherein the first and second materials differ from each other. The first material may include a first deodorizing agent and a carrier, and the second material may include a second deodorizing agent and a carrier, wherein the first deodorizing agent, second deodorizing agent, and carrier are selected from the compositions noted above. Without limitation, the first material may consist, consist essentially of, or include a combination of a long chain fatty acid such as stearic acid with a carrier material such as paraffin wax, soy wax and/or cetearyl alcohol, and the second material may consist, consist essentially of, or include a combination of a metallic salt such as zinc ricinoleate and a carrier such as paraffin wax, soy wax, and/or cetearyl alcohol. The first portion 1260-1 may include a combination of stearic acid and a carrier material (e.g., paraffin wax). The second portion 1260-2 may include zinc ricinoleate in range of 0.5 to 20 weight %, the remainder of second portion 1260-2 being a carrier material (e.g., paraffin wax) in a range of 80 to 99.5 weight %.

The first portion 1260-1 and the second portion 1260-2 can be configured to couple together and form a single monolithic structure. The first portion 1260-1 and the second portion 1260-2 can be coupled together via, for instance, an adhesive coupling, a mechanical coupling, and/or any other form of coupling.

The deodorizer 1208 may have an overall volume in a range of 20 ml to 80 ml. The deodorizer 1208 may have an overall height in a range of 5 mm to 25 mm, an overall length in a range of 100 mm to 280 mm and an overall thickness in a range of 5 mm to 25 mm.

The first portion 1260-1 and the second portion 1260-2 can include identical or different dimensions. The first portion 1260-1 and the second portion 1260-2 can include the same or different shape/profiles.

The first portion 1260-1 may have an overall volume in a range of 10 ml to 70 ml. The first portion 1260-1 may have an overall height in a range of 5 mm to 25 mm, an overall length in a range of 100 mm to 280 mm, and an overall thickness in a range of 2.5 mm to 20 mm.

The second portion 1260-2 may have an overall height in a range of 5 mm to 25 mm, an overall length in a range of 100 mm to 280 mm, and an overall thickness in a range of 2.5 mm to 20 mm.

As discussed above, the first portion 1260-1 can be formed using a process similar to the process discussed above with regard to FIG. 11. The second portion 1260-2 can be formed by melting a deodorizing agent and carrier together. This melt can be either poured into a separate mold to cool in a similar way to forming the first portion 1260-1 or poured into the same mold as the first portion 1260-1 (e.g., over the top of the solidified/cooled first portion).

The example of FIG. 12 may also be referred to as a multi-action strip/bar. The multi-action strip may include at least two different portions/sections such as the first portion 1260-1 and the second portion 1260-2. The at least two different sections can be coupled together and form an interface therebetween. Forming the deodorizer 1208 can include forming each section separately and allowing each to cool, e.g., to room temperature. The two cooled sections may then be coupled together as shown in FIG. 12. Alternatively, a first section may be formed and cooled. Once cooled, a second section may then be disposed on the first section and cooled.

The deodorizer 1208 can include a multi-action configuration where each section targets a different group/classification of malodor molecules. For example, the first portion 1260-1 targets molecules containing sulfur, aldehydes, ketones and alcohols based on the long chain fatty acid, e.g., stearic acid, and the second portion 1260-2 targets molecules containing carboxylic acid based on the odor eliminating compound, e.g., zinc ricinoleate.

In a scenario where stearic acid is included in the first portion 1260-1 and zinc ricinoleate is included in the second portion 1260-2, the interface may advantageously isolate each section to limit the deactivation of the zinc ricinoleate to the region forming the interface. As is known, zinc ricinoleate can be deactivated based on a chemical interaction of the carboxylic acid head of the stearic acid binding/using up active sites in the zinc ricinoleate.

In operation, each section/portion of the deodorizer 1208 is in contact (direct) with the surface to be cleaned and/or an associated cleaning element, such as the second cleaning element 310-2 (sec, FIG. 3).

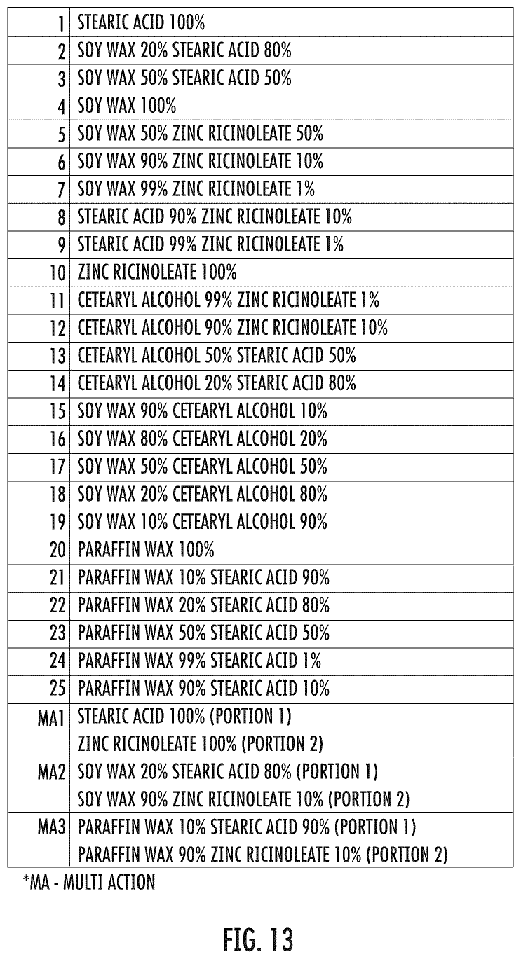

FIG. 13 shows example material combinations for the deodorizer 1108 (sec, combinations 1-25) and for the multi-action (MA) configuration of the deodorizer 1208.

One particular formulation that was found to be particularly well suited for forming a deodorizing composition odor was 80 weight % Steric Acid with 20 weight % Soy Wax, combined with 10% Zinc Ricinoleate and 90% Soy Wax.

Selection of a formulation for a deodorizer consistent with the present disclosure may provide one or more of the following: a deodorizer that can be mechanically engaged to output a predetermined amount of particles (e.g., in a range of 0.01 to 1.00 grams per hour for at least fifteen hours of operation of a cleaning device); a deodorizer that is chemically stable in water; a deodorizer that is safe to contact human skin and stable in contact with household surfaces; a deodorizer that does not chemically react to household cleaning chemicals such as vinegar or bleach; a deodorizer whose released particles will not cause damage to a surface to be cleaned and, should the particles accumulate on a surface, those cumulative particles do not negatively impact the look or feel of the surface, such as a carpet, nor damage floor surfaces such as hard woods, e.g., by de-stabilizing floor glue/applicator; and/or a deodorizer whose released particles will not damage parts/components within the surface cleaning device and/or cause discoloration/staining.

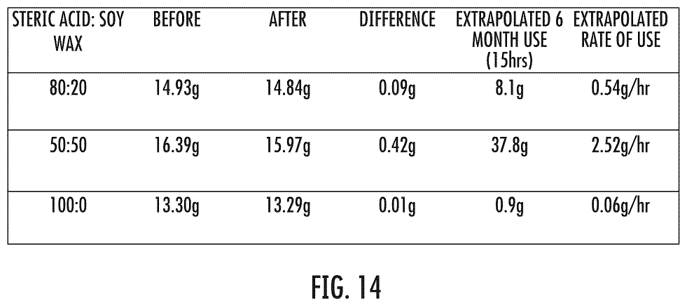

Deodorizers consistent with the present disclosure were tested with different compound configurations as shown in the table of FIG. 14 to determine relative durability. Strips/bars with a greater concentration of stearic acid were found to be generally harder/rigid and therefore demonstrated a relatively high amount of durability relative to other compound configurations.

Each test sequence included the following steps: (1) weigh strip/bar to determine initial weight in grams (g); (2) place strip/bar into direct contact with soft brush bar (or foam roller implemented as the second cleaning element 310-2 in FIG. 3) ensuring contact between the strip/bar and soft brush bar or foam roller remains throughout the duration of the test. This test can be performed with either gravity and/or a spring member exerting a force (F1, FIG. 3) on the strip/bar. In the example shown in table in FIG. 14, a spring force was used and supplied a bias force; (3) operate surface cleaning device for ten minutes, during which the surface cleaning device remains stationary and the associated cleaning element is rotated while in direct contact with the strip/bar (e.g., see FIG. 3); (4) weigh the strip/bar after the ten minutes, and note new weight in grams; (5) determine overall weight loss in grams by subtracting the new weight from the original weight; (6) extrapolate difference between initial weight and weight after ten minutes of use to determine six month loss value and hourly rate of use/loss values.

Example compounds for use within a deodorizer consistent with the present disclosure include zinc ricinoleate, Dipalmitoylethyl hydroxyethylmonium methosulfate (Cationic surfactant) to increase substantivity to fabric (e.g., how much it sticks to fabric/carpet), fragrance fixatives such as triethyl citrate, diphenylmethane, and/or a low odor fragrance chemical such as citral or limonene.

Various features and aspects of the present disclosure may be implemented in both powered and unpowered surface cleaning devices. Powered surface cleaning devices refer to a surface cleaner configured to drive/energize at least one cleaning element, such as a suction motor and/or brush roll, based on a DC and/or AC power signal. Some such examples of powered surface cleaning devices include robotic vacuums and cannister vacuums.

Unpowered surface cleaning devices refer to surface cleaning devices that perform a cleaning operation without generating an electrical load, e.g., on a battery or AC mains. Such unpowered surface cleaning devices can include a carpet sweeper that utilizes a mechanical assembly to collect debris based on a user-supplied force (or manual force) that causes forward/backward movement of the carpet sweeper against a surface to be cleaned.

The present disclosure also recognizes that in some scenarios a powered surface cleaning device having a deodorizer consistent with the present disclosure may be used in an unpowered/manual mode when odor management is desired. For instance, a surface cleaning device consistent with the present disclosure may be pushed/pulled by a user when the surface cleaning device is powered off/deenergized to cause the odor member to be mechanically engaged via the surface to be cleaned and/or cleaning clement (e.g., a brush roll). This mechanical engagement can cause the deodorizer to release particles onto the surface to be cleaned and/or into the surrounding area for odor management without requiring battery power to be consumed, or requiring the surface cleaning device to be plugged into an electrical outlet via a power cord.

An example of a surface cleaning device, consistent with the present disclosure, may include a nozzle, a dust cup, a suction motor configured to draw air into the nozzle and through the dust cup, and a first deodorizer coupled to the nozzle, the first deodorizer includes a deodorizing composition having a long chain fatty acid.

In some instances, the long chain fatty acid may be a stearic acid. In some instances, the deodorizing composition may be 50 to 100 weight % stearic acid. In some instances, the first deodorizer may include a first portion formed of the long chain fatty acids and a second portion formed of an odor control compound. In some instances, the odor control compound may include zinc ricinoleate. In some instances, the deodorizing composition may include a carrier material. In some instances, the carrier material may include paraffin wax. In some instances, the first deodorizer may be configured to directly engage a surface to be cleaned during cleaning operations. In some instances, the nozzle may include a cleaning element and the first deodorizer engages the cleaning element. In some instances, the cleaning element may be a foam roller. In some instances, a second deodorizer may be coupled to the nozzle. In some instances, at least one of the first deodorizer or the second deodorizer may be configured to directly engage a surface to be cleaned. In some instances, the nozzle may include a cleaning element and at least one of the first deodorizer or the second deodorizer is configured to directly engage the cleaning element. In some instances, the cleaning element may be a brush roll or a foam roller. In some instances, the first deodorizer may be removably coupled to the nozzle. In some instances, the first deodorizer may include a cartridge body. In some instances, the nozzle may include a cartridge receptacle configured to receive the cartridge body. In some instances, the cartridge receptacle may include a latch configured to releasably engage the cartridge body. In some instances, surface cleaning device may include further include a plurality of cleaning elements. In some instances, at least one cleaning element may be different from at least one other cleaning element.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. It will be appreciated by a person skilled in the art that a surface cleaning apparatus may embody any one or more of the features contained herein and that the features may be used in any particular combination or sub-combination. Modifications and substitutions by one of ordinary skill in the art are considered to be within the scope of the present disclosure, which is not to be limited except by the claims.

What is claimed is:

1. A surface cleaning device comprising:
a nozzle;
a first agitator rotatably coupled to the nozzle, the first agitator configured to rotate about a first longitudinal axis; and
a first deodorizer coupled to the nozzle and configured to contact an outer surface of the first agitator as the first agitator rotates about the first longitudinal axis.

2. The surface cleaning device of claim 1, wherein the first agitator is a foam roller.

3. The surface cleaning device of claim 1, further comprising a second deodorizer coupled to the nozzle.

4. The surface cleaning device of claim 3, wherein the second deodorizer is configured to directly engage a surface to be cleaned.

5. The surface cleaning device of claim 1, wherein the first deodorizer is removably coupled to the nozzle.

6. The surface cleaning device of claim 5, wherein the first deodorizer includes a cartridge body.

7. The surface cleaning device of claim 6, wherein the nozzle includes a cartridge receptacle configured to receive the cartridge body.

8. The surface cleaning device of claim 7, wherein the cartridge receptacle includes a latch configured to releasably engage the cartridge body.

9. The surface cleaning device of claim 1 further comprising a second agitator.

10. The surface cleaning device of claim 9, wherein the first agitator is different from the second agitator.

11. The surface cleaning device of claim 9, further comprising:
a dust cup; and
a suction motor;
the nozzle further including a front side, a back side, and an opening on an underside of the nozzle between the front side and the back side, the opening fluidly coupled to the suction motor;
wherein the second agitator is mounted to the nozzle within the suction conduit and configured to rotate about a second longitudinal axis; and
wherein the first agitator is closer to the front of the nozzle than the second agitator.

12. The surface cleaning device of claim 1, wherein the first agitator is a foam roller and the second agitator is a brush roll.

13. The surface cleaning device of claim 1, wherein the first deodorizer includes an engagement surface configured to directly contact the outer surface of the first agitator.

14. The surface cleaning device of claim 13, wherein the engagement surface includes a profile that corresponds with a profile of the first agitator.

15. The surface cleaning device of claim 1, wherein the surface cleaning device includes a robotic vacuum.

16. A surface cleaning device comprising:
a nozzle;
a first agitator rotatably coupled to the nozzle, the first agitator configured to rotate about a first longitudinal axis;
a second agitator rotatably coupled to the nozzle, the second agitator configured to rotate about a second longitudinal axis which is parallel to the first longitudinal axis;
a first deodorizer coupled to the nozzle and configured to contact an outer surface of the first agitator as the first agitator rotates about the first longitudinal axis.

17. The surface cleaning device of claim 16, wherein the nozzle includes a front region, a back region, and an opening on an underside of the nozzle between the front region and the back region, and wherein the first agitator is located closer to the front region than the second agitator is to the front region.

18. The surface cleaning device of claim 17, wherein the first agitator includes a foam roller.

19. The surface cleaning device of claim 18, wherein the second agitator includes a plurality of bristles.

20. The surface cleaning device of claim 17, wherein the first agitator includes a soft brush bar.
